(19) United States
(12) Patent Application Publication
(10) Pub. No.: US 2008/0319370 A1
(43) Pub. Date: Dec. 25, 2008

(54) Method and System for Transdermal Drug Delivery

(75) Inventors: John Wolpert, New South Wales (AU); Nina Frances Wilkins, Coburg (AU)

Correspondence Address: FOLEY AND LARDNER LLP;SUITE 500, 3000 K STREET NW, WASHINGTON, DC, 20007, US

(73) Assignee: Acrux DDS Pty Ltd.

(21) Appl. No.: 12/092,452

(22) PCT Filed: Nov. 3, 2006
(86) PCT No.: PCT/AU06/01635

§ 371 Date: Jul. 11, 2008

(30) Foreign Application Priority Data
Nov. 4, 2005 (AU) 2005906134

(51) Int. Cl. A61N 1/30

(52) U.S. Cl. 604/20

(57) ABSTRACT

The present invention relates to a method and a system for transdermal delivery of a physiologically active agent. The invention further relates to a method of treatment using the transdermal delivery system and the use of medicaments (5) from manufacture of such as system.

This Application claims priority from Australian Provisional Patent Application No 2005906134 (4 Nov. 2005) the contents of which are herein incorporated by reference.

### FIELD

The present invention relates to a method and a system for transdermal delivery of a physiologically active agent. The invention further relates to a method of treatment using the transdermal delivery system and the use of medicaments from manufacture of such a system.

### BACKGROUND

Rapid transdermal delivery of a physiologically active agent would offer several clinical and patient advantages in the treatment of the symptoms of conditions or disease.

Drug delivery by injection is traditionally the quickest route of administration to the systemic circulation, however the duration of action is often short lived and the mode of delivery invasive and painful. Transdermal drug delivery is receiving increased attention due to the ability of the administration regime to provide a controlled route for the release of an active agent to the systemic circulation. However, transdermal drug delivery is complicated by the fact that the skin behaves as a natural barrier. As a result, the success of transdermal delivery systems often relies on the ability of a composition to be able to penetrate the stratum corneum of the skin and thereby transport an active agent across the skin.

It is desirable to reduce the time required for transdermal delivery of a physiologically active agent across the skin, so that rapid onset of beneficial effects can be achieved. Transdermal delivery also has the potential to provide sustained and controlled drug delivery.

In order to improve the rate of transdermal delivery of an active agent, a number of methods have been used. One method involves the use of microneedles to breach the stratum corneum, resulting in the formation of micropores in the skin. The micropores facilitate the delivery of a therapeutic composition directly to the viable epidermis. Devices incorporating microneedles often include a reservoir, which provides a supply of the drug is to be administered transdermally. When the microneedles breach the stratum corneum, the drugs are then fed from the reservoir to the microneedles by a lumen within the needle itself or from the underside of an occlusive device such as a patch.

Another method that has been used to improve the rate of transdermal delivery is sonophoresis. Using this technique, cavitation is produced when ultrasound induced pressure variation is applied to the skin.

Ionotophoresis has also been used to assist in transdermal delivery of a physiologically active agent. This method involves the application of galvanic or direct electrical current, which then gives rise to charged ions of an active agent, which are propelled through the skin.

The application of heat to the area of skin application of a transdermal composition using heated bandages or the like has also been reported.

The present invention seeks to improve transdermal delivery of physiologically active agents by utilising electroporation techniques. This technique involves the application of short and rapid pulses of electrical energy to induce the formation of openings or holes in the skin. Devices that utilise electroporation to facilitate transdermal delivery have been described, however such devices typically combine a pulse generator and filament grid together with a reservoir of the active agent in an occlusive system, such as a patch.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

There is a need for enhanced transdermal administration of a transdermal or topical composition into the skin and/or systemic circulation where rapid onset is desirable.

### SUMMARY

The present invention relates to a method and system for improved transdermal administration of a physiologically active agent. The method and system of the present invention utilises the technique of electroporation to increase the rate of flux of an active across the skin and thereby allow rapid onset of beneficial effects from the active to be achieved.

In one aspect, the present invention provides a method for transdermal delivery of a topically applied physiologically active agent, wherein the method comprises the steps of:
(i) applying a pulse of electrical energy to an area of the skin; and
(ii) contacting the area of the skin with a topical transdermal composition comprising at least one physiologically active agent, a pharmaceutically acceptable carrier and optionally at least one dermal penetration enhancer.

We have found that the combination of electrical energy and penetration enhancer significantly enhance the uptake of the physiologically active agent.

In another aspect, the present invention provides a system for transdermal delivery of a topically applied physiologically active agent, the system comprising:
(i) a container for holding a transdermal composition comprising at least one physiologically active agent, a pharmaceutically acceptable carrier and optionally, at least one dermal penetration enhancer;
(ii) an applicator for applying the transdermal composition to the skin of a subject from the container;
(iii) a source of electricity;
(iv) a skin contacting element in electrical communication with the source of electricity; and
(v) an actuator connected to the source of electricity, said actuator being operable to cause a pulse of electrical energy to be transmitted to the skin contacting element

The system may be occlusive, however it is preferred that the system be non-occlusive.

The applicator may deliver the composition in a suitable manner, such as a roll-on, spray, aerosol, capillary delivery, brush, swab or other suitable means.

The actuator may be operably coupled to the applicator to co-ordinate application of the transdermal composition and delivery of a pulse of electricity. This is particularly preferred for spray application. Alternatively, though less preferably, the transdermal delivery device may comprise a separate actuator for application of the transdermal composition.

The delivery device may be used to apply a pulse of electrical energy to an area of the skin through which transdermal penetration of the active is to occur before, during or after the application of the transdermal composition.

Throughout the description and the claims of this specification the word “comprise” and variations of the word, such as “comprising” and “comprises” is not intended to exclude other additives, components, integers or steps.

### DETAILED DESCRIPTION

The rate of transdermal uptake of drugs and other physiologically active agents is generally considered to be slow. The rate of transdermal administration may be assisted by the use of dermal penetration enhancers, which provide a non-invasive yet effective method of improving drug delivery across the skin.

The use of a source of electrical energy for the ablation of tissue in a technique known as electroporation facilitates the transdermal delivery of physiologically active agents and provides a means by which improved percutaneous absorption efficiency of a physiologically active agent may be achieved.

Electroporation uses high-voltage, short duration pulses, which are thought to create localised regions of permeable membrane by producing aqueous pathways in lipid membrane bilayers (Tsong T. (1991). Electroporation of cell membranes. Biophys. J. 60:297-306). This routine tool for destabilising has been the subject of increasing focus as a means to enhance transdermal transport (Prausnitz M. R, Bose V G, Langer R, Weaver J C (1993), Electroporation of mammalian skin: a mechanism to enhance transdermal drug delivery. Proc. Natl. Acad. Sci. USA. 90:10504-10508). Electroporation of the stratum corneum in vitro is typically performed using square wave or exponential voltage pulses.

In one aspect, the present invention provides a method for transdermal delivery of a topically applied physiologically active agent, wherein the method comprises the steps of:
(i) applying a pulse of electrical energy to an area of the skin; and
(ii) contacting the area of the skin with a topical transdermal composition comprising at least one physiologically active agent, a pharmaceutically acceptable carrier and optionally at least one dermal penetration enhancer.

In applying a pulse of electrical energy to an area of the skin, the electrical energy is believed to create localised regions of permeable membrane by producing aqueous pathways in lipid membrane bilayers. Without being limited by theory, the electrical pulse subsequently creates microscopic openings or holes in the skin. Microscopic openings generated in the skin by the pulse of electrical energy can facilitate the rapid delivery of a physiologically active agent across the skin. However, the microscopic openings created in the skin may close after a period of approximately 2 minutes. Therefore, the inclusion of a penetration enhancer may enable sustained or enhanced systemic delivery.

The pulse of electrical energy may be generated by any suitable means that is able to provide an electricity source. In a preferred embodiment, the source of electricity is a battery. In a more preferred embodiment, the source of electricity is a capacitor or a transformer.

The pulse of electrical energy is of sufficient voltage and duration to facilitate the transdermal administration of a physiologically active agent through the skin of a subject. Preferably, a pulse of electricity energy is of the order of between 0.3 kV and 8 kV. More preferably, the pulse of electricity energy is of the order of approximately 1 kV. It is also preferred that the pulse of electrical energy be of relatively short duration. In this regard, an electrical energy pulse may be between 0.1 to 20 milliseconds. An electrical energy pulse of approximately 10 milliseconds duration is most preferred. It would be appreciated by the person skilled in the art that the voltage and duration required could be varied to suit the particular application and for the delivery of different dosages of the active agent. The method of the present invention also envisages the ability to apply repeated pulses of electrical energy to the surface of the skin in order to enable repeated or sustained transdermal delivery of the physiologically active agent across the skin. Accordingly, the ability to control the dosage regime in this manner may be desirable when it is desired to deliver a large quantity of the active initially, then provide a lower, sustained dosage over time.

The method of the present invention also comprises the step of contacting the area of the skin of a subject, such as a human or animal, with a transdermal composition. In this respect, the transdermal composition is generally topically applied to the surface of the skin. The transdermal composition may be applied to an area of the skin before, during or after the application of electrical energy to the area of the skin. The transdermal composition and the electrical energy are generally applied to the same area of skin of a subject.

The topical transdermal composition comprises at least one physiologically active agent and a pharmaceutically acceptable carrier. The transdermal composition used in accordance with the present invention may and preferably will comprise at least one dermal penetration enhancer in addition to the physiologically active agent. The presence of a dermal penetration enhancer may assist to provide additional increases in the rate of transport of an active agent across the skin, thereby enhancing uptake of the active agent into the systemic circulation via the vasculature or lymphatic system. There may however, be some circumstances in which the enhancer is not necessary for the required treatment or dose of active agent.

The method of the present invention preferably involves the application of a single dose of the transdermal composition to an area of the skin. However, it may also be necessary to apply a number of dosages to the same or different areas of the skin to obtain the desired result.

The method of the present invention advantageously enables the physiologically active agent from the transdermal composition to be released into the systemic circulation such that a rapid first order release rate profile can be achieved and maintained. The release rate profile preferably reaches maximum rate of flux within 6 hours, more preferably within 1 hour. Because of the efficiency of the method of the invention, the dosage of the physiologically active agent may often be less than that conventionally used and the surface area of application may be reduced. Furthermore, with the time to Tmax significantly reduced, the invention would be particularly useful for therapies that require rapid onset or acute use, such as pain relief. Since the present invention creates microscopic openings in the skin, it is most likely that compounds normally considered inappropriate for transdermal delivery, such as those with large molecular weights and certain physicochemical characteristics, may be delivered across the skin.

The method of the present invention enables the rapid onset of beneficial effects for the treatment of humans, however the method of the invention can also be extended to the treatment of non-human animals.

Surprisingly, it has been found that a large range of physiologically active agents can be rapidly delivered to the systemic circulation of a subject by the methods of the present invention.

In another aspect, the present invention provides a system for transdermal delivery of a topically applied physiologically active agent, the system comprising:
(i) a container for holding a topical transdermal composition comprising at least one physiologically active agent, a pharmaceutically acceptable carrier and optionally, at least one dermal penetration enhancer;
(ii) an applicator for applying the transdermal composition to the skin of a subject;
(iii) a source of electricity;
(iv) a skin contacting element in electrical communication with the source of electricity; and
(v) an actuator connected to the source of electricity, said actuator being operable to cause a pulse of electrical energy to be transmitted to the skin contacting element

The system of the present invention may be occlusive, however it is preferred that the system be non-occlusive. In this regard, non-occlusive systems preferably do not require a patch or other occlusive element in order to achieve the desired advantages.

The system of the present invention includes a transdermal delivery device. The transdermal delivery device is preferably hand-held so as to allow ease of use and convenience to the user of the device.

The transdermal delivery device includes a container for holding a topical transdermal composition comprising at least one physiologically active agent, a pharmaceutically acceptable carrier and optionally, at least one dermal penetration enhancer. The container stores the transdermal composition until it is desired for application to an area of the skin of a subject. The container may be any suitable container, and a number of commercially available plastic and/or glass containers that are suitable for pharmaceutical formulations will suffice. Preferably, the container is connected to an actuator of the delivery device, so that upon activation of the actuator, a quantity of the transdermal composition may be dispensed from the container.

The transdermal delivery device also includes an applicator. The applicator is used to apply the topical transdermal composition to the skin of a subject. A number of types of suitable applicator may be used to deliver the composition in a suitable manner. Suitable types of applicator include for example, roll-ons, sprays, aerosols, capillary delivery systems, brushes, swabs, pump-packs or other applicators. It is particularly preferred that the applicator is a spray applicator.

Preferably, the applicator provides either a fixed or variable metered dose application. Examples include a metered dose aerosol, a stored-energy metered dose pump or a manual metered dose pump. A preferred transdermal delivery device is described in co-pending US Patent Application Publication No. 2004/0050964, incorporated herein by reference, wherein the topical metered dose aerosol is combined with an actuator nozzle shroud which together accurately control the amount and/or uniformity of the dose applied.

The transdermal delivery device also includes a source of electricity. It is preferred that the source of electricity may be any suitable source and is preferably a portable source, such as a battery, capacitor or a transformer.

The transdermal delivery device includes an actuator. At least one actuator is included in the device, although two or more actuators may be present. The actuator is preferably connected to the source of electricity, such that upon activation of the actuator, for example, by depression of the actuator, a pulse of electrical energy is generated from the source of electricity. The same actuator may be in communication with the container of the transdermal delivery device, so that the transdermal composition is dispensed from the container upon activation of the actuator and applied to the skin of a subject. The transdermal composition may be dispensed upon activation of the same actuator that is in communication with the source of electricity. Alternatively, the composition may be dispensed upon activation of a different actuator provided by the same applicator.

In a preferred embodiment, the transdermal composition is propelled from the transdermal delivery device by the use of propellants such as hydrocarbons, hydrofluorocarbons, nitrogen, nitrous oxide, carbon dioxide or ethers. Preferably, the propellant is dimethyl ether or HFC 134a.

The transdermal delivery device also includes a skin contacting element in electrical communication with the means for providing a source of electricity. The pulse of electrical energy generated by the source of electricity may therefore be conducted to the skin contacting element. Without being limited by theory, it is believed that the electrical pulse produces an aqueous pathway in the lipid membrane bilayer in the skin. As a result, upon the skin contacting element being contacted with the skin of a subject, microscopic openings may be generated in the skin, which may facilitate the transdermal delivery of an active agent across the skin.

The skin contacting element generally comprises an array of filaments. The array of filaments may assist to generate a pulse with consistent contact across the area of skin. The skin contacting element may be of any suitable shape and configuration that achieves the advantages of the present invention. In one preferred embodiment, the skin contacting element consists of a disc having a grid-like arrangement of filaments. In another preferred embodiment, the skin contacting element consists of a retractable iris configuration, with the filaments in a concentric arrangement. The iris may open or close to allow the transdermal composition and/or electrical energy to be applied to the surface of the skin. Preferably, the filaments are metallic.

The transdermal composition comprises at least one systemic or locally acting physiologically active agent or prodrug thereof. The transdermal composition is preferably a single phase system as this allows less complicated manufacture and ease of dose uniformity. The transdermal composition may also include combinations of two or more physiologically active agents.

Physiologically active agents that may be used in the percutaneous or transdermal drug delivery system of the present invention include any locally or systemically active agents which are compatible with the dermal penetration enhancers of the present invention and which can be delivered through the skin with the assistance of the dermal penetration enhancer to achieve a desired effect. These active agents (grouped by therapeutic class) include:

### Cardiovascular System

Antihypertensives such as hydralazine, minoxidil, captopril, enalapril, clonidine, prazosin, debrisoquine, diazoxide, guanethidne, methyldopa, reserpine, trimetaphan. Calcium channel blockers such as diltiazem, felodopine, amlodipine, nitrendipine, nifedipine and verapamil.

Antiangina agents such as glyceryl trinitrate, erythritol tetranitrate, pentaerythritol tetranitrate, mannitol hexanitrate, perhexylene, isosorbide dinitrate and nicorandil.

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine. Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol.

Antimigraine preparations such as ergotamine, dihydroergotamine, methysergide, pizotifen and sumatriptan.

Drugs Affecting Blood and Haemopoietic Tissues.

Anticoagulants and thrombolytic agents such as warfarin, dicoumarol, low molecular weight heparins such as enoxaparin; streptokinase and its active derivatives. Haemostatic agents such as aprotinin, tranexamic acid and protamine.

### Central Nervous System

Analgesics, antipyretics including the opiod analgesics-such as buprenorphine, dextromoramide, dextropropoxyphene, fentanyl, alfentanil, sufentanil, hydromorphone, methadone, morphine, oxycodone, papavereturn, pentazocine, pethidine, phenoperidine, codeine and dihydrocodeine. Others include acetylsalicylic acid (aspirin), paracetamol, and phenazone.

Hypnotics and sedatives such as the barbiturates, amylobarbitone, butobarbitone and pentobarbitone and other hypnotics and sedatives such as choral hydrate, chlormethiazole, hydroxyzine and meprobamate.

Antianxiety agents such as the benzodiazepines, alprazolam, bromazepam, chlordiazepoxide, clobazam, chlorazepate, diazepam, flunitrazepam, flurazepam, lorazepam, nitrazepam, oxazepam, temazepam and triazolam. Neuroleptic and antipsychotic drugs such as the phenothiazines, chlorpromazine, fluphenazine, pericyazine, perphenazine, promazine, thiopropazate, thioridazine and trifluoperazine and the butyrophenones, droperidol and haloperidol and the other antipsychotic drugs such as pimozide, thiothixene and lithium.

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline.

CNS stimulants such as caffeine.

Anti-alzheimer's agents such as tacrine.

Antiemetics, antinauseants such as the phenothiazines, prochloperazine, thiethylperazine and 5HT-3 receptor antagonists such as ondansetron and granisetron and others such as dimenhydrinate, diphenhydramine, metoclopramide, domperidone, hyoscine, hyoscine hydrobromide, hyoscine hydrochloride, clebopride and brompride.

### Musculoskeletal System

Non-steroidal anti-inflammatory agents including their racemic mixtures or individual enantiomers where applicable, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol and ketoralac.

Additional non-steroidal antiinflammatory agents which can be formulated in combination with the dermal penetration enhancers include salicylamide, salicylic acid, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloide, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Muscle relaxants such as baclofen, diazepam, cyclobenzaprine hydrochloride, dantrolene, methocarbamol, orphenadrine and quinine.

### Hormones and Steroids

Oestrogens such as oestradiol, oestriol, oestrone, ethinyloestradiol, mestranol, stilboestrol, dienoestrol, epioestriol, estropipate and zeranol. Progesterone and progestins (ie. progestagens other than progesterone) such as allyloestrenol, dydrogesterone, lynoestrenol, norgestrel, norethyndrel, norethisterone, norethisterone acetate, gestodene, levonorgestrel, nestorone, methoxyprogesterone and megestrol.

The hormones may be contraceptive hormones such as at least one progestin such as norethindrone, norethindrone acetate, norethisterone acetate, gestodene, desogestrel drospirenone, ethynodiol diacetate, norelgestromin, norgestimate, levonorgestrel, nestorone, methoxyprogesterone, megestrol and dl-norgestrel, optionally in combination with one or more oestrogens such as oestradiol, oestriol, oestrone, ethinyloestradiol, mestranol, stilboestrol, dienoestrol, epioestriol, estropipate and zeranol.

Antiandrogens such as cyproterone acetate and danazol.

Antioestrogens such as tamoxifen and epitiostanol and the aromatase inhibitors, exemestane and 4-hydroxy-androstenedione and its derivatives.

Androgens and anabolic agents such as testosterone, methyltestosterone, clostebol acetate, drostanolone, furazabol, nandrolone oxandrolone, stanozolol, trenbolone acetate, dihydro-testosterone, 17-α-methyl-19-nortestosterone and fluoxymesterone.

5-alpha reductase inhibitors such as finasteride, turosteride, LY-191704 and MK-306.

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide.

Further examples of steroidal antiinflammatory agents for use in the instant compositions include include cortodoxone, fluoracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

Pituitary hormones and their active derivatives or analogs such as corticotrophin, thyrotropin, follicle stimulating hormone (FSH), luteinising hormone (LH) and gonadotrophin releasing hormone (GnRH).

Hypoglycaemic agents such as insulin, chlorpropamide, glibenclamide, gliclazide, glipizide, tolazamide, tolbutamide and metformin.

Thyroid hormones such as calcitonin, thyroxine and liothyronine and antithyroid agents such as carbimazole and propylthiouracil.

Other miscelaneous hormone agents such as octreotide.

Pituitary inhibitors such as bromocriptine.

Ovulation inducers such as clomiphene.

### Genitourinary System

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene.

Antidiuretics such as desmopressin, lypressin and vasopressin including their active derivatives or analogs.

Obstetric drugs including agents acting on the uterus such as ergometrine, oxytocin and gemeprost.

Prostaglandins such as alprostadil (PGE1), prostacyclin (PGI2), dinoprost (prostaglandin F2-alpha) and misoprostol.

### Antimicrobials

Antimicrobials including the cephalosporins such as cephalexin, cefoxytin and cephalothin.

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, mezlocillin, piperacillin, ticarcillin and azlocillin.

Tetracyclines such as minocycline, chlortetracycline, tetracycline, demeclocycline, doxycycline, methacycline and oxytetracycline and other tetracycline-type antibiotics.

Aminoglycosides such as amikacin, gentamicin, kanamycin, neomycin, netilmicin and tobramycin.

Antifungais such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione.

Quinolones such as nalidixic acid, cinoxacin, ciprofloxacin, enoxacin and norfloxacin. Sulphonamides such as phthalylsulphthiazole, sulfadoxine, sulphadiazine, sulphamethizole and sulphamethoxazole.

Sulphones such as dapsone.

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, timidazole, fusidic acid and trimethoprim; 2-thiopyridine N-oxide; halogen compounds, particularly iodine and iodine compounds such as iodine-PVP complex and diiodohydroxyquin; hexachlorophene; chlorhexidine; chloroamine compounds; benzoylperoxide.

Antituberculosis drugs such as ethambutol, isoniazid, pyrazinamide, rifampicin and clofazimine.

Antimalarials such as primaquine, pyrimethamine, chloroquine, hydroxychloroquine, quinine, mefloquine and halofantrine.

Antiviral agents such as acyclovir and acyclovir prodrugs, famciclovir, zidovudine, didanosine, stavudine, lamivudine, zalcitabine, saquinavir, indinavir, ritonavir, n-docosanol, tromantadine and idoxuridine.

Anthelmintics such as mebendazole, thiabendazole, niclosamide, praziquantel, pyrantel embonate and diethylcarbamazine.

Cytotoxic agents such as plicamycin, cyclophosphamide, dacarbazine, fluorouracil and its prodrugs [described, for example, in International Journal of Pharmaceutics 111, 223-233 (1994)], methotrexate, procarbazine, 6-mercaptopurine and mucophenolic acid.

### Metabolism

Anorectic and weight reducing agents including dexfenfluramine, fenfluramine, diethylpropion, mazindol and phentermine.

Agents used in hypercalcaemia such as calcitriol, dihydrotachysterol and their active derivatives or analogs.

### Respiratory System

Antitussives such as ethylmorphine, dextromethorphan and pholcodine. Expectorants such as acetylcysteine, bromhexine, emetine, guaiphenesin, ipecacuanha ans saponins.

Decongestants such as phenylephrine, phenylpropanolamine ans pseudoephedrine.

Bronchospasm relaxants such as ephedrine, fenoterol, orciprenaline, rimiterol, salbutamol, sodium cromoglycate, cromoglycic acid and its prodrugs [described, for example, in International Journal of Pharmaceutics 7, 63-75 (1980)], terbutaline, ipratropium bromide, salmeterol and theophylline and theophylline derivatives.

### Allergy and Immune System

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

Local anaesthetics such as bupivacaine, amethocaine, lignocaine, cinchocaine, dibucaine, mepivacaine, prilocalne and etidocaine.

Stratum corneum lipids, such as ceramides, cholesterol and free fatty acids, for improved skin barrier repair [Man, et al. J. Invest. Dermatol., 106(5), 1096, 1996].

Neuromuscular blocking agents such as suxamethonium, alcuronium, pancuronium, atracurium, gallamine, tubocurarine and vecuronium.

Smoking cessation agents such as nicotine, bupropion and ibogaine.

Insecticides and other pesticides which are suitable for local or systemic application.

Dermatological agents, such as vitamins A and E, vitamin E acetate and vitamin E sorbate.

Allergens for desensitization such as house dust mite allergen.

Nutritional agents, such as vitamins, essential amino acids and essential fats.

Keratolytics such as the alpha-hydroxy acids, glycollic acid and salicylic acid.

Psychicenergisers, such as 3-(2-aminopropyl)indole, 3-(2-aminobutyl)indole, and the like.

Anti-acne agents such as containing isotretinoin, tretinoin and benzoyl peroxide.

Anti-psoriasis agents such as containing etretinate, cyclosporin and calcipotriol.

Anti-itch agents such as capsaicin and its derivatives such as nonivamide [Tsai, et al. Drug. Dev. Ind. Pharm., 20(4), 719, 1994].

Anticholinergic agents, which are effective for the inhibition of axillary sweating and for the control of prickly heat. The antiperspirrant activity of agents such as methatropine nitrate, propantheline bromide, scopolamine, methscopolamine bromide, and the new class of soft antiperspirants, quaternary acyloxymethyl ammonium salts [described, for example, by Bodor et al, J. Med. chem. 23, 474 (1980) and also in United Kingdom Specification No. 2010270, published Jun. 27, 1979].

Other physiologically active peptides and proteins, small to medium-sized peptides, e.g., vasopressin and human growth hormone.

The composition of the invention is applied to the skin of an animal. Preferably the animal is a human but the invention also extends to the treatment of non-human animals.

Preferably the non-occlusive drug delivery system is not supersaturated with respect to the physiologically active agent or prodrug. As the volatile liquid of the non-occlusive drug delivery system evaporates, the resulting non-volatile composition is rapidly driven into the dermal surface or mucosal membrane. It is possible that as the volatile liquid evaporates, the non-volatile dermal penetration enhancer becomes supersaturated with respect to the active agent. However, it is preferred that any supersaturation does not occur before transport of the resulting non-volatile composition across the epidermal surface

Preferred physiologically active agents include, but are not limited to, macromolecules and hormones such as insulin, ACTH (corticotropin), parathyroid hormone, growth hormone (GH) and its analogues, GH antagonists, luteinizing hormone releasing hormone, follicle stimulating hormone, G-CSF, heparin, monoclonal antibodies, DNA polymers, genes and oligonucleotides, alpha-1 anti trypsin, anti-angiogenesis agents, anti-sense agents, butorphanol, calcitonin and its analogues, ceredase, COX-II inhibitors, dermatological agents, dihydroergotamine, dopamine agonists and antagonists, opioid peptides, hormones such as testosterone, analgesics including narcotic analgesics such as fentanyl, sufentanil, oligosaccharides, prostaglandins, sildenafil, thrombolytics, tissue plasminogen activators, RNF, vaccines, anti-tuberculosis agents, anti-addiction agents, anti-allergy agents, antiemetics and antinauseants such as granisetron and ondansetron, anti-obesity agents, anti-osteoporotics, anti-infectives, anaesthetics, anorexics, antiarthritics, antiasthmatic agents such as terbutaline, anticonvulsants, anti-depressants, anti-diabetic agents, antihistamines, anti-inflammatory agents including non-steroidal anti-inflammatory agents, anti-migraine agents, antineoplastics, antiParkinsonians, antipruritics including corticosteroids, antipsychotics, antipyretics, anticholinergics, benzodiazepine antagonists, vasodilators, antivirals, adrenergic stimulants such as adrenaline, ephedrine, terbutaline, dobutamine, phenylephrine, phenylpropanolamine, pseudoephedrine and dopamine, antianxiety agents, anti-Alzheimer's agents.

The amount of physiologically active agent in the transdermal composition and administered to a subject, will vary from subject to subject and will depend on a number of factors, including for example, the particular physiologically active agent administered, the severity of the symptoms, the subject's age, weight and general condition, and the judgment of the prescribing physician. The minimum amount of physiologically active agent is determined by the requirement that sufficient quantities of the drug must be present in the composition to maintain the desired rate of release over the given period of application. The maximum amount for safety purposes is determined by the requirement that the quantity of drug present cannot exceed a rage of release that reaches toxic levels. Generally, the maximum concentration is determined by the amount of agent that can be received without producing adverse histological effects such as irritation. Of course it will be appreciated by those skilled in the art that the desired dose of a specific drug will depend on the nature of the drug as well as on other factors; the minimum effective dose of each physiologically active agent is of course preferred.

The transdermal composition also comprises a pharmaceutically acceptable carrier. The carrier is generally required to be compatible with the physiologically active agent and allows the composition comprising the active agent to be topically applied to the skin. Suitable carriers would be apparent to the person skilled in the art. In a preferred embodiment, the carrier may be a safe skin-tolerant volatile liquid. These are preferably used in the range 50 to 99%

In systems according to the present invention, an adjuvant such as a pharmaceutical compounding agent, co-solvent, surfactant, emulsifier, antioxidant, preservative, stabiliser, diluent or a mixture of two or more of said components may be incorporated in the transdermal composition in appropriate amounts to the particular dosage form. The amount and type of adjuvants used should be compatible with the active ingredient and other possible components of the transdermal composition. A co-solvent or other standard adjuvant, such as a surfactant, may be required to maintain the agent in solution or suspension at the desired concentration.

The pharmaceutical compounding agents can include paraffin oils, esters such as isopropyl myristate, ethanol, silicone oils and vegetable oils. These are preferably used in the range 1 to 50%. Surfactants such as ethoxylated fatty alcohols, glycerol mono stearate, phosphate esters, and other commonly used emulsifiers and surfactants preferably in the range of 0.1 to 10% may be used, as may be preservatives such as hydroxybenzoate esters for preservation of the compound preferably in amounts of 0.01% to 0.5%. Typical co-solvents and adjuvants may be ethyl alcohol, isopropyl alcohol, acetone, dimethyl ether and glycol ethers such as diethylene glycol mono ethyl ether. These may be used in amounts of from 1 to 50%.

The transdermal composition may optionally also include at least one dermal penetration enhancer. In this regard, dermal penetration enhancers are generally adapted to transport the physiologically active agent across a dermal surface or mucosal membrane of an animal, including a human. The dermal penetration enhancer is preferably of low toxicity to, and is tolerated by, the dermal surface or mucosal membrane of the animal. The dermal penetration enhancer may be selected from the classes of enhancers that are lipophilic non-volatile liquids whose vapour pressure is below 10 mm Hg at atmospheric pressure and normal skin temperature of 32 degrees Celsius. Preferably, the dermal penetration enhancer has a molecular weight within the range of 200 to 400 Daltons. Examples of suitable dermal penetration enhancers include: laurocapram (Azone®) and laurocapram derivatives, such as those 1-alkylazacycloheptan-2-ones specified in U.S. Pat. No. 5,196,410, and oleic acid and its ester derivatives, such as methyl, ethyl, propyl, isopropyl, butyl, vinyl and glycerylmonooleate, and sorbitan esters such as sorbitan monolaurate and sorbitan monooleate, and other fatty acid esters such as isopropyl laurate, isopropyl myristate, isopropyl palmitate, diisopropyl adipate, propylene glycol monolaurate and propylene glycol monooleate, and long chain alkyl esters of 2-pyrrolidone, particularly the 1-lauryl, 1-hexyl and 1-(2-ethylhexyl)esters of 2-pyrollidene and the sunscreen ester dermal penetration enhancers disclosed in U.S. Pat. No. 6,299,900: and the dermal penetration enhancers disclosed in U.S. Pat. No. 5,082,866, particularly dodecyl(N,N-dimethylamino)acetate and dodecyl(N,N-dimethylamino) propionate and in U.S. Pat. No. 4,861,764, particularly 2-n-nonyl-1-3-dioxolane.

A particularly preferred group of penetration enhancers are selected from the group consisting of C₈ to C₁₈ alkylcinnamate, C₈ to C₁₈ alkyl methoxycinnamate and C₈ to C₁₈ alkyl salicylate. The most preferred are octyl salicylate and octyl para-methoxycinnamate (Padimate O).

The concentration of dermal penetration enhancer may be in the range from 0.1-30% of the total composition. The ratio of penetration enhancer to active ingredient may vary considerably and will be governed as much as anything, by the pharmacological results that are required to be achieved. In principle, it is desirable that as little penetration enhancer as possible is used. On the other hand, for some actives, it may well be that the upper range of 10,000% by weight will be required. It is preferred that the penetration enhancer is in the range of 0.1-10%.

The concentration of physiologically active agent used in the transdermal composition will depend on its properties and may be equivalent to that normally utilised for the particular agent in conventional formulations. Both the amount of physiologically active agent and the amount of penetration enhancer will be influenced by the type of effect desired. For example, if a more localised effect is required in treating a superficial infection with an antibacterial agent, lower amounts of physiologically active agents and lower concentrations of enhancer may be appropriate. Where deeper penetration is desired, as in the case of local anaesthesia, a higher concentration of enhancer may be appropriate.

Where it is desired to achieve systemic concentration of an agent, proportionately higher concentrations of the enhancer of the invention may be required in the transdermal composition and system of the present invention, and the amount of active substance included in the composition should be sufficient to provide the blood level desired.

In a preferred embodiment, the system of the present invention is non-occlusive, and the transdermal composition preferably comprises:
(i) an effective amount of at least one physiologically active agent or prodrug thereof;
(ii) at least one non-volatile dermal penetration enhancer; and
(iii) at least one volatile liquid.

In a preferred embodiment, the dermal penetration enhancer is adapted to transport the physiologically active agent across the skin when the volatile liquid evaporates, such that a reservoir or depot of a mixture comprising the penetration enhancer and the physiologically active agent or prodrug is therefore formed within the skin.

In order to achieve this effect, the volatile liquid carrier preferably has a vapour pressure above 35 mm Hg at atmospheric pressure and normal skin temperature of 32 degrees Celsius. In a particularly preferred form of the invention the volatile liquid is selected from ethanol, ethyl acetate or isopropanol, or mixtures thereof in the range of about 50 to 99%. An aerosol propellant, such as dimethyl ether or a hydrofluorocarbon (HFC) such as the hydrofluoroalkane HCF-134a may also constitute a volatile liquid for the purpose of the present invention.

Preferably, the transdermal composition is not supersaturated with respect to the physiologically active agent or prodrug. As the volatile liquid of the non-occlusive system evaporates, the resulting non-volatile composition is rapidly driven into the dermal surface or mucosal membrane. It is possible that as the volatile liquid evaporates, the non-volatile dermal penetration enhancer becomes supersaturated with respect to the active agent. However, it is preferred that any supersaturation does not occur before transport of the resulting non-volatile composition across the epidermal surface has occurred.

It is most desirable that, after application of the non-occlusive, percutaneous or transdermal delivery system, the volatile component of the delivery system evaporates and the area of skin to which the drug delivery system was applied becomes touch-dry upon application of 100 μl spread evenly across the skin surface. Preferably said area of skin becomes touch-dry within 10 minutes, more preferably within 3 minutes, most preferably within 1 minute.

In a preferred embodiment of the non-occlusive system of the present invention, whilst a transdermal composition comprising adjuvants such as pharmaceutical compounding agents, co-solvents, surfactants, emulsifiers, antioxidants, preservatives, stabilisers, diluents or a mixture of two or more of said components may be used, it is particularly preferred that the adjuvants be selected so as to be compatible with the ability of the system becoming touch-dry after application of the composition to an area of the skin.

### BRIEF DESCRIPTION OF THE FIGURES

FIG. 1 shows a graph illustrating the effect of the application of a voltage of 1 kV for 10 milliseconds on the permeation of fentanyl through the human epidermis.

FIG. 2 shows an exploded view of a system in accordance with one aspect of the present invention.

FIG. 3 shows a cross-sectional view of the system of FIG. 2.

FIG. 4 shows a plan view of a skin contacting element having a filament arranged as a grill for use in the system of FIG. 2.

FIG. 5 shows a cross-sectional view of the skin contacting element of FIG. 4 in which the electrical contact filaments are shown.

FIG. 6 shows a plan view of a skin contacting element having a retractable iris arrangement for use in the system of FIG. 2.

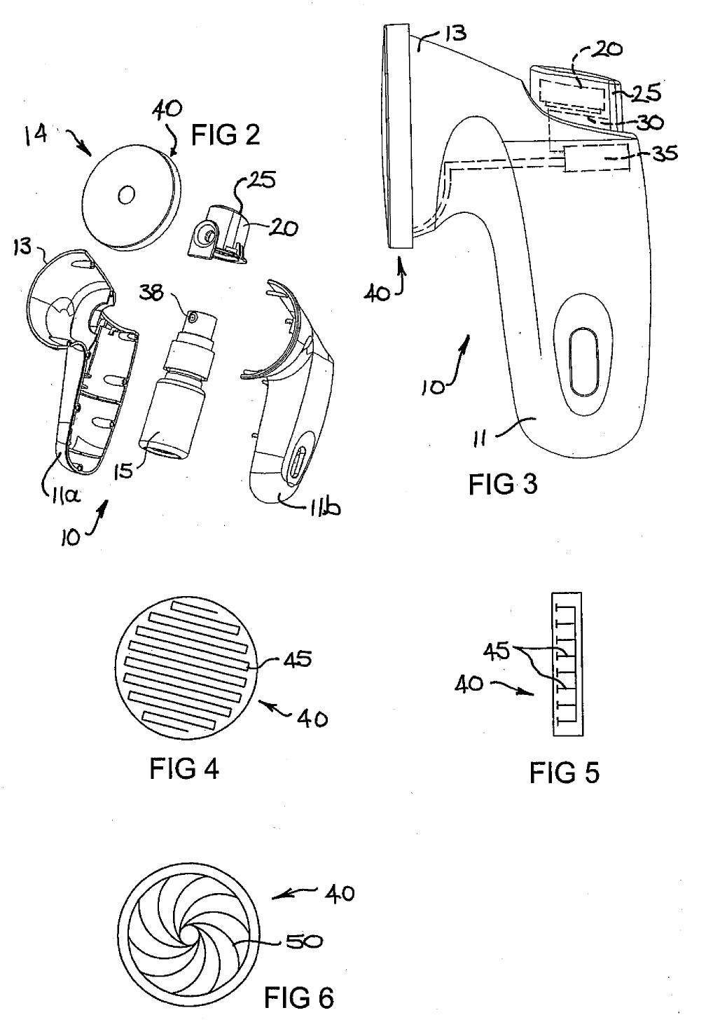

Referring now to FIGS. 2 and 3, a system in accordance with one aspect of the present invention is shown. The system comprises a spray applicator device (10) having a bottle (15) for containing a transdermal composition comprising at least one physiologically active agent, a pharmaceutically acceptable carrier and optionally at least one dermal penetration enhancer.

The spray applicator device (10) includes a hollow body (11) formed from two separable parts (11a, 11b). These parts combine to form the hollow body (11) when they are connected together. By connecting the parts around the bottle (15), the hollow body (11) is thereby able to house the bottle (15) within. The parts (11a, 11b) may be releasably connected by any suitable arrangement, such as for example, a pin and hole arrangement.

The spray applicator device (10) also includes a shroud (13), which may be covered by a lid (14). The shroud (13) can assist in ensuring that the transdermal composition is confined to the target area after the composition has been delivered by the spray applicator (10). The shroud (13) is preferably substantially conical in form. The wider portion of the shroud (13) is placed adjacent the skin of a subject when the applicator (10) is in use.

The spray applicator device (10) also includes a battery (20) for providing a source of electricity. The battery (20) is connected to an actuator button (25) of the applicator device (10). Upon activation of the actuator button (25) by the user depressing the button (25), the battery (20) connects with contact points (30), to transmit a pulse of energy from the battery (20) to a pulse generator (35) housed within the spray applicator (10). The pulse generator (35) then produces a pulse of electrical energy.

The actuator button (25) also co-operates with the bottle (15) containing the transdermal composition to deliver a quantity or dose of the composition. In this regard, the actuator button (25) may co-operate with a pump, so that when the pump is operated upon depression of the actuator (25), a quantity of the composition is withdrawn from the bottle (15) and expelled from the spray applicator (10) via a spray nozzle (38) for delivery to the skin of a subject. Thus in this manner, the same actuator button (25) is used to generate the pulse of electrical energy as well as deliver the transdermal composition.

The spray applicator device (10) includes a skin contacting element (40) in electrical communication with the pulse generator (35) and the battery (20). The skin contacting element (40) is located at the outlet area of the shroud (13). In use, the pulse of electrical energy transmitted to the pulse generator (35) is conducted to the skin contacting element (40).

With reference to FIG. 4, a skin contacting element (40) in accordance with one embodiment of the invention is shown. The skin contacting element (40) may consist of a disc having a filament (45) arranged in a grille, through which the transdermal composition and/or energy can pass once the actuator (25) has been activated. With reference to FIG. 5, a

.filament (45) may traverse a cross-sectional area of the skin contacting element (4) and assist to conduct the generated electrical energy from the spray applicator device (10) to the skin of a subject.

Referring now to FIG. 6, a skin contacting element (40) in accordance with another embodiment of the present invention is shown. In this embodiment, the skin contacting element (40) consists of a retractable iris configuration comprised of a series of concentric filaments (50). The filaments (50) of the iris may open and close upon activation of the actuator (25) to thereby allow the transdermal composition and/or heat energy to be applied to the surface of the skin.

In describing the present invention, the following terminology will be used in accordance with the definitions set out below.

The term “stratum corneum” is used herein in its broadest sense to refer to the outer layer of skin, which is comprised of (approximately 15) layers of terminally differentiated keratinocytes made primarily of the proteinaceous material keratin arranged in a ‘brick and mortar’ fashion, with the mortar being comprised of a lipid matrix made primarily from cholesterol, ceramides and long chain fatty acids. The stratum corneum creates the rate-limiting barrier for diffusion of the active agent across the skin.

The term “dermal penetration enhancer” is used herein in its broadest sense to refer to an agent which improves the rate of percutaneous transport of active agents across the skin for use and delivery of active agents to organisms such as animals, whether it be for local application or systemic delivery.

The term “physiologically active agent” is used herein to refer to a broad class of useful chemical and therapeutic agents.

The term “physiologically active” in describing the agents contemplated herein is used in a broad sense to comprehend not only agents having a direct pharmacological effect on the host, but also those having an indirect or observable effect which is useful in the medical arts.

It is believed that the rate of initial uptake of the physiologically active agent into the systemic circulation is rapidly enhanced by the application.

The invention will now be described with reference to the following examples. It is to be understood that the examples are provided by way of illustration of the invention and that they are in no way limiting to the scope of the invention.

### EXAMPLES

Study Treatments

| | | |
|---|---|---|
| A | Fentanyl | 5% |
| | Octyl salicylate | 5% |
| | IPA | to volume |
| B | Fentanyl | 5% |
| | IPA | to volume |

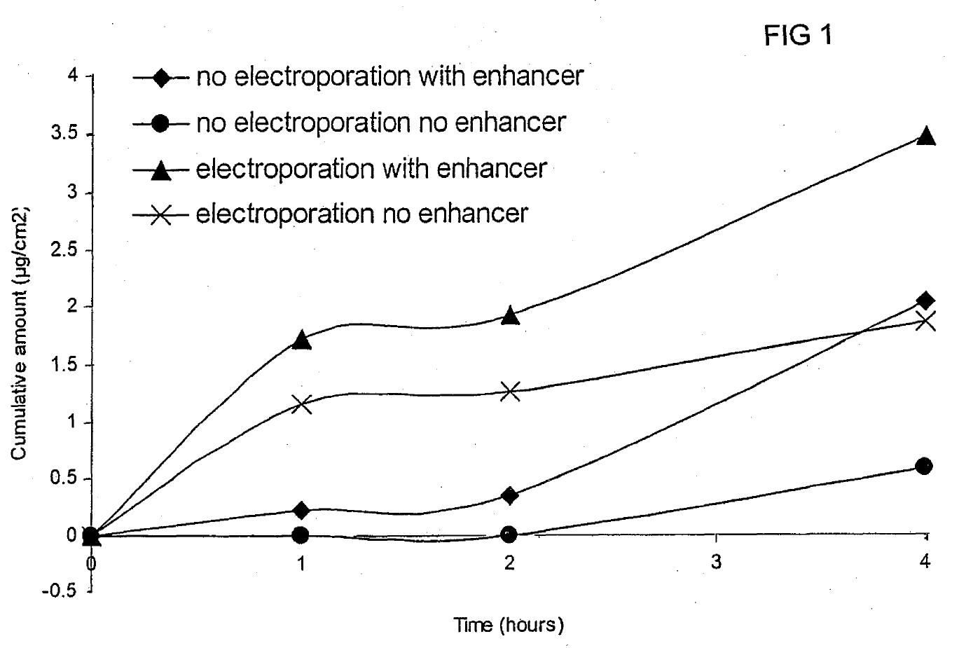

As shown in FIG. 1, the combination of short duration, high voltage electric currents and the addition of a penetration enhancer on human skin permeation of fentanyl prior to dose application resulted in a significant effect on shortening lag time.

The diffusion experiments were performed using human epidermis as the model membrane. The epidermis was backed onto filter paper for additional support. These experiments were performed over 24 h with stainless steel, flow-through diffusion cells based on those previously described, (Cooper, E. R. J. Pharm. Sci. 1984, 73, 1153-1156.) except that the cell was modified to increase the diffusional area to 1.0 cm². A High Voltage (1 kV) was applied to the skin for 10 mS, immediately prior to dosing. A finite dose of 5 μl/cm² of either formulation A or B was applied to the diffusion cell and left uncovered for the diffusion of the experiment. A piece of stainless steel wire mesh was placed directly below the skin in the receptor chamber of the diffusion cell to maintain a turbulent flow of receptor solution below the skin. The diffusion cells were maintained at a flow rate of approximately 1.0 mL/cm²/h by a microcassette peristaltic pump (Watson Marlow 505S, UK). The cells were kept at 32±0.5° C. by a heater bar and the samples are collected into appropriately sized plastic vials on an automated fraction collector (Isco Retriever II, Lincoln, Nebr.) at specified intervals. The receptor solution (20% EtOH with 0.002% sodium azide) maintained sink conditions beneath the skin.

Samples were analysed by RP-HPLC using the following conditions; Column-Waters Symmetry C₁₈ column (3.9×150 mm) with a 5 μm support size; Mobile phase-80% ACN in aqueous 0.009% PCA with 9 mM 1-HAS, 20% AcN; Flow rate-1.0 mL/min; Absorbance-210 nm; and Injection volume-50 μL

It is understood that various other modifications and/or alterations may be made without departing from the spirit of the present invention as outlined herein.

1. A method for transdermal delivery of a topically applied physiologically active agent, wherein the method comprises the steps of:
(i) applying a pulse of electrical energy to an area of the skin; and
(ii) contacting the area of the skin with a topical transdermal composition comprising at least one physiologically active agent, a pharmaceutically acceptable carrier and optionally at least one dermal penetration enhancer.

2. A method according to claim 1 wherein the pulse of electrical energy is between about 0.3 kV and 8 kV.

3. A method according to claim 1 wherein the pulse of electrical energy is about 1 kV.

4. A method according to claim 1 wherein the topical transdermal composition is applied to an area of the skin before, during or after the application of the pulse of electrical energy.

5. A method according to claim 1 wherein the transdermal composition is applied from an applicator by roll-on, spray, aerosol, capillary delivery, brush, swab.

6. A method according to claim 5 wherein the actuator may be operably coupled to the applicator to co-ordinate application of the transdermal composition and delivery of a pulse of electricity.

7. A method according to claim 1 wherein the composition comprises at least one non volatile dermal penetration enhancer.

8. A method according to claim 7 wherein the transdermal penetration enhancer comprises at least one selected from the group consisting of laurocapram (Azone®) and laurocapram derivatives, oleic acid; fatty acid esters, long chain alkyl esters of 2-pyrrolidone and sunscreen ester penetration enhancers.

9. A method according to claim 7 wherein the composition comprises a sunscreen ester penetration enhancer selected from octyl salicylate, octyl dimethyl para-aminobenzoate and octyl para-methoxycinnamate (Padimate 0).

10. A method according to claim 7 wherein the total amount of non volatile penetration enhancer is in the range of from 0.1 to 10% by weight of the total composition.

11. A method according to claim 1 wherein the composition comprises at least one volatile liquid selected from ethanol, ethyl acetate or isopropanol, or mixtures thereof in a total amount of volatile liquid in the range of from about 50 to about 99% by weight of the total composition.

12. A system for transdermal delivery of a topically applied physiologically active agent, the system comprising:
(i) a container;
(ii) a transdermal composition within the container comprising at least one physiologically active agent, a pharmaceutically acceptable carrier and optionally, at least one dermal penetration enhancer;
(iii) an applicator for applying the transdermal composition to the skin of a subject;
(iv) a source of electricity;
(v) a skin contacting element in electrical communication with the source of electricity; and
(vi) an actuator connected to the means for providing a source of electricity, said actuator being operable to cause a pulse of electrical energy to be transmitted to the skin contacting element.

13. A system according to claim 12 which is non-occlusive.

14. A system according to claim 12 wherein the skin contacting element comprises one or more filaments.

15. A system according to claim 14 wherein the one or more filaments form a grid.

16. A system according to claim 14 wherein the skin contacting element is a retractable iris and a plurality of filaments are arranged concentrically about the iris.

17. A system according to claim 12 wherein the applicator is in the form of a roll-on, spray applicator, aerosol, capillary delivery applicator, brush or swab.

18. A system according to claim 17 wherein the applicator is an aerosol or a spray applicator.

19. A system according to claim 12 wherein the actuator is operably coupled to the applicator to co-ordinate application of the transdermal composition and delivery of a pulse of electricity.

20. A system according to claim 12 wherein the container is connected to an actuator of the delivery device, so that upon activation of the actuator, a quantity of the transdermal composition is be dispensed from the container.

21. A system according to claim 12 wherein the container comprises a metered dose aerosol, a stored-energy metered dose pump or a manual metered dose pump.

22. A system according to claim 12 wherein the transdermal composition is propelled from the transdermal delivery device by a propellant comprising at least one of hydrocarbons, hydrofluorocarbons, nitrogen, nitrous oxide and carbon dioxide.

23. A system according to claim 12 wherein the transdermal composition comprises at least one dermal penetration enhancer.

24. A system according to claim 23 wherein the at least on dermal penetration enhancer is selected from the group consisting of laurocapram (Azone®), laurocapram derivatives, oleic acid, fatty acid esters, long chain alkyl esters of 2-pyrrolidone, and sunscreen ester penetration enhancers.

25. A system according to claim 23 wherein the penetration enhancer is selected from the group consisting of octyl salicylate, octyl dimethyl para-aminobenzoate and octyl para-methoxycinnamate (Padimate 0).

26. A system according to claim 12 wherein the carrier comprises a skin-tolerant volatile liquid in an amount in the range of from 50 to 99% by weight.

27. A system according to claim 12 wherein the penetration enhancer is present in an amount in the range of 0.1-10% by weight of the transdermal composition.

28. A system according claim 12 wherein the carrier comprises a volatile liquid is selected from ethanol, ethyl acetate or isopropanol, or mixtures thereof in an amount to provide a total volatile solvent component in the range of from about 50 to 99%.
